(19) United States
(12) Patent Application Publication
(10) Pub. No.: US 2022/0118043 A1
(43) Pub. Date: Apr. 21, 2022

(54) Hair Loss Treatment Composition

(71) Applicant: Brittany Bonds, Indianapolis, IN (US)

(72) Inventor: Brittany Bonds, Indianapolis, IN (US)

(21) Appl. No.: 17/476,789

(22) Filed: Sep. 16, 2021

Related U.S. Application Data
(60) Provisional application No. 63/094,477, filed on Oct. 21, 2020.

(51) Int. Cl. A61K 36/87; A61K 31/047; A61K 36/752; A61K 31/192; A61K 31/4166; A61K 9/08; A61K 9/00; A61K 31/07; A61K 31/375

(52) U.S. Cl. CPC A61K 36/87; A61K 31/047; A61K 36/752; A61K 31/192; A61K 31/375; A61K 9/08; A61K 9/0014; A61K 31/07; A61K 31/4166

(57) ABSTRACT

This present invention relates to a hair treatment spray which be used to promote hair strength, durability, and growth. The hair treatment spray comprises of all-natural products including grapeseed oil, watermelon extract, orange extract, pineapple extract, lime oil, Vitamins A, C, and E, and more, and is free from harmful chemicals and parabens which inhibits from additional hair damage due to conventional hair care products. The hair treatment spray can be used by people of all ages and for any hair color and type, which reconstructs and rejuvenates the hair follicle and scalp in order to restore hair growth and inhibit further hair loss

### CROSS-REFERENCE TO RELATED APPLICATION

The present application claims priority to, and the benefit of, U.S. Provisional Application No. 63/094,477, which was filed on Oct. 21, 2020 and is incorporated herein by reference in its entirety.

### FIELD OF THE INVENTION

The present invention relates generally to the field of hair treatment. More specifically, the present invention relates to an all-natural hair spray to promote hair strength, durability and growth. The all-natural hair spray utilizes water-based ingredients combined with different oils, vitamins and extracts to create the hair loss treatment composition. The hair loss treatment composition improves overall hair health to repair damage, thereby ensuring hair grows healthy and long. The hair treatment composition can be in the form of a spray, and can be applied as an additional step while washing and conditioning hair. The hair spray of the present invention is easy to use and can be used by people of all ages, and for any hair color and type. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, various people suffer from hair health issues such as hair loss, hair dullness and other conditions due to poor and stunted hair growth. People around the world are concerned when they suffer from hair loss or alopecia, or other similar issues. Various conditions such as over-processed hair, stress, medical side effects, hormonal changes, scalp infections, medical conditions and other health reasons can cause damage to hair and eventually cause hair loss. Other conditions such as a dry and itchy scalp can cause individuals to scratch their head, which eventually injures hair follicles, leading to hair breakage and hair care complications. People with hair health problems can get frustrated and seek out hair treatment products to prevent further damage to their hair.

Alopecia may occur in various parts of the human body, such as the scalp, face, limbs, and trunk, and may be generally grouped in the following categories: male or female pattern baldness (e.g., androgenic alopecia), toxic alopecia (e.g., hair loss due to chemotherapy), alopecia aerata (i.e., hair loss occurring in demarcated areas), and scarring alopecia (e.g., hair loss due to inflammation and tissue destruction). Androgenic alopecia is one of the most common causes of hair loss and it occurs due to a deficiency in androgenic metabolism, e.g., accumulation of dihydrotestosterone (DHT) in hair follicles, owed to a defective regulation in an enzyme responsible for DHT synthesis.

Various solutions are available in the market for people suffering from hair health issues. For example, shampoos, hair lotions, hair oils, hair conditioners or other similar products are specially manufactured to strengthen the hair and hair follicles of individuals. Although, such products claim to cure hair loss, strengthen and thicken the hair, and condition the scalp, none of the currently available products are effective. These products are usually comprised of harmful chemicals and parabens which cause a number of additional problems such as drying of hair, irritation to scalp, hair loss and more. A lack of effective options exist to treat hair loss and dryness, particularly options that provide a natural solution. Further, some people may seek medical help to overcome their hair health problems. However, frequent multiple visits to healthcare centers, medical treatments and travel to healthcare centers can be both expensive and time-consuming.

Therefore, there exists a long felt need in the art for a hair treatment product that inhibits hair loss and hair breakage problems. There is also a long felt need in the art for a hair care product that is effective in the treatment of conditions such as hair loss, hair dullness, poor hair growth and other similar conditions. Additionally, there is a long felt need in the art for a hair treatment product that is free from chemicals and parabens. Moreover, there is a long felt need in the art for a hair treatment product that does not cause additional problems of dry scalp, itchy scalp or more. Further, there is a long felt need in the art for a hair care product that is manufactured using natural ingredients. Furthermore, there is a long felt need in the art for a hair treatment product that eliminates the need for individuals to seek medical help, and eliminates the unwanted expenses due to medical treatment. Finally, there is a long felt need in the art for a cost-effective hair product that is all natural and promotes hair strength, durability and growth.

The subject matter disclosed and claimed herein, in one embodiment thereof, comprises a natural hair care spray composition combined with distilled water or other carrier-based fluid. The composition comprises glycerin, grapeseed oil, watermelon extract, orange extract, pineapple extract, lime oil, Vitamins A, C, and E, flavonoids, phenolic compounds, ferulic acid and allantoin to improve overall hair health to repair damage, thereby ensuring hair grows healthy and long. The spray composition functions as a hair care product for people of all ages and for any hair color and type and reconstructs and rejuvenates the hair follicles and the scalp in order to restore hair growth and inhibit further hair loss.

In this manner, the novel all-natural hair care product of the present invention accomplishes all of the forgoing objectives, and provides a relatively safe, easy, cost-effective and effective solution to promote hair strength, durability and growth. The all-natural ingredient hair care product of the present invention is also user friendly, inasmuch as it is less expensive than other products heretofore known. The hair care product of the present invention eliminates the use of harmful chemicals and parabens. Additionally, the all-natural hair care product is easy to use and can be applied while washing or conditioning hair.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a natural hair care spray composition and distilled water or other carrier base fluid. The composition comprises glycerin, grapeseed oil, watermelon extract, orange extract, pineapple extract, lime oil, Vitamins A, C, and E, flavonoids, phenolic compounds, ferulic acid and allantoin to improve overall hair health. The spray composition functions as a hair care product for people of all ages and for any hair color and type, and reconstructs and rejuvenates the hair follicles and the scalp in order to restore hair growth and inhibit further hair loss.

In a further embodiment of the present invention, a method of treating hair loss and conditioning hair of both men and women is disclosed and comprises the steps of contacting an individual's hair and scalp where hair growth is desired with a hair strengthening composition comprising: 3-6% glycerin, 0.3-10% grapeseed oil, 3-5% watermelon extract, 2-14% orange extract, 2-24% pineapple extract, 3-15% lime oil, 0.2-3% natural vitamins A, C, and E, 0.1-2% flavonoids, 0.5-1.8% phenolic compounds, 0.3-2.2% ferulic acid, 0.5-0.8% allantoin and distilled water.

In a further embodiment of the present invention, a composition for spray for use in the treatment of hair loss comprises, by weight of the composition, 4-5% glycerin, 1-5% grapeseed oil, 4-8% watermelon extract, 3-18% orange extract, 3-20% pineapple extract, 4-8% lime oil, 1-2.5% natural vitamins A, C, and E, 1-2% flavonoids, 0.8-1.5% phenolic compounds, 0.4-2.1% ferulic acid, 0.5-0.8% allantoin and distilled water.

The composition of the present invention does not require animal testing. The composition does not include sulfates, gluten, paraffin, parabens, phthalates, DEA or artificial coloring. The composition is readily absorbed into the scalp tissue. The composition is a safe all-natural composition for hair growth and stimulation wherein the composition has been derived from naturally occurring sources.

The advantage of the composition of the present invention is that it provides users with an all-natural hair spray designed to promote hair strength, durability, and growth. The ingredients act in harmony with each other to achieve the desired results of scalp repair and hair growth. The spray treatment can be applied at room temperature and provides relaxation, cooling and moisture to the skin surface of the scalp. Further, the composition can also promote beard growth for men treating their facial hair. A user can apply a small amount of the composition in the form of a spray to their hair every day. Upon applying, the composition absorbs into the scalp and hair follicle shaft.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of the ingredients of the hair loss treatment composition spray of the present invention used to treat hair loss, dry scalp, and other hair-related issues;

FIG. 2 illustrates an image of a user prior to using the hair loss treatment composition spray of the present invention;

FIG. 3 illustrates an image of the same user after using the hair loss treatment composition spray of the present invention for a month;

FIG. 4 illustrates an exemplary pump spray container in which the hair loss treatment composition spray can be dispensed and/or sold as per the present embodiment of the present invention; and

FIG. 5 illustrates components of a kit in which the hair loss treatment composition spray can be marketed and commercially made available.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

Hair is composed of a strong structural protein called keratin. Hair forms in a pouch-like structure below the skin called a hair follicle, which encloses the hair root. Visible hair, such as scalp hair for example, is the hair shaft, which is keratinized, hardened tissue that grows from the hair follicle. At the base of the follicle is the dermal papilla. The dermal papilla is fed by the bloodstream which carries nourishment to produce new hair. The dermal papilla plays a crucial role in the dermal-epidermal interactions and is of great importance for hair formation.

Throughout the life of an individual, hair growth and hair renewal are determined by the activity of the hair follicles. Normal hair follicles cycle between a growth stage (anagen), a degenerative stage (catagen), and a resting stage (telogen), which are each characterized by very specific molecular and cellular mechanisms. Scalp hairs have a relatively long life cycle. The anagen stage can range from 2 to 6 years; the catagen stage can range from a few days to a few weeks; and, the telogen stage lasts approximately three months. Shorter hairs found elsewhere on the human body have corresponding shorter anagen durations. The morphology of the hair and the hair follicle changes dramatically over the course of the life cycle of the hair.

During the anagen phase the hair follicle is highly active metabolically. The follicle comprises a dermal papilla at the base of the follicle; and epidermal matrix cells surrounding the dermal papilla form the base of the hair shaft, which extends upwards from the papilla through the hair canal. The matrix cells are the actively growing portion of the hair. The cells of the dermal papilla “send” signals to the matrix cells, which then migrate to the hair follicle matrix. In this region, the cells of the dermal papilla emit additional signals which allow the matrix cells to proliferate and then to differentiate, further allowing the elongation of the hair shaft. It is believed that hair follicles are formed only in the early stages of development and are not replaced. Thus, an increase in damaged or non-functioning hair follicles is generally associated with hair loss. It is further believed that hair growth may be restored to a dormant and/or damaged hair follicle.

As noted above, there exists a long felt need in the art for a hair treatment product that inhibits hair loss and hair breakage problems. There is also a long felt need in the art for a hair care product that is effective in the treatment of conditions such as hair loss, hair dullness, poor hair growth and other similar conditions. Additionally, there is a long felt need in the art for a hair treatment product that is free from chemicals and parabens. Moreover, there is a long felt need in the art for a hair treatment product that does not cause additional problems such as dry scalp, itchy scalp or more. Further, there is a long felt need in the art for a hair care product that is manufactured using natural ingredients. Finally, there is a long felt need in the art for a cost-effective hair product that is all natural and promotes hair strength, durability and growth.

The present invention, in one exemplary embodiment, is a novel, natural hair care spray composition comprising distilled water or carrier base fluid, glycerin, grapeseed oil, watermelon extract, orange extract, pineapple extract, lime oil, Vitamins A, C, and E, flavonoids, phenolic compounds, ferulic acid and allantoin to improve overall hair health and to repair damage. The spray composition functions as a hair care product for people of all ages and for any hair color and type and reconstructs and rejuvenates the hair follicles and scalp in order to restore hair growth and inhibit further hair loss.

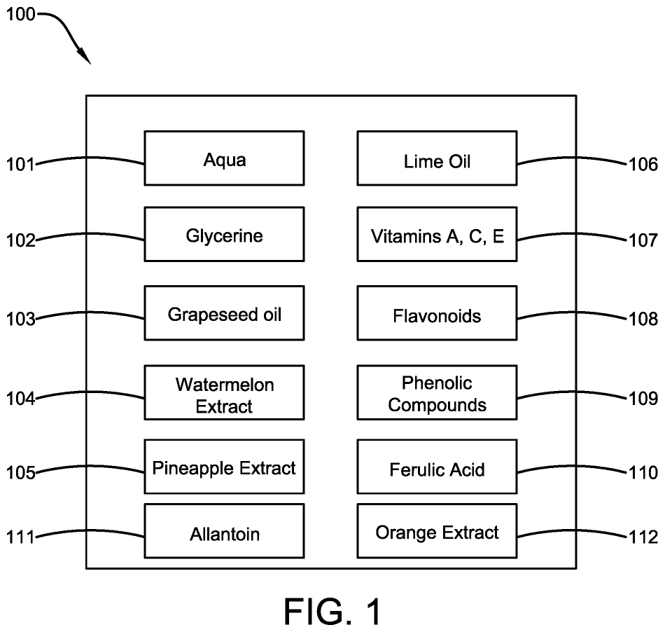

Referring initially to the drawings, FIG. 1 illustrates a diagrammatic representation of the ingredients of the hair loss treatment composition spray of the present invention used to treat hair loss, dry scalp and other hair-related issues. The hair loss treatment composition spray 100 of the present invention is made up of natural ingredients which are derived from commercial sources. All the ingredients provide a unique quality and increase the effectiveness of the hair loss treatment composition spray 100 in promoting hair strength, durability, growth and improved overall hair health.

Aqua 101 in the form of distilled water (i.e. carrier base fluid) forms the solvent for the hair loss treatment composition spray 100. The composition spray 100 can have the form of a solution or dispersion in distilled water 101. Glycerin 102 provides natural conditioning and hydration to hair and helps to lock in moisture. Glycerin further reduces itching, inhibits hair loss and stimulates hair growth.

Grapeseed oil 103 acts as a natural dandruff treatment and improves the condition of hair and scalp. Also, grapeseed oil 103 includes linoleic fatty acid which provides hydration to hair. Furthermore, the flavonoid ‘oligomeric procyanidin’ found in grape seed oil is an incredibly potent antioxidant. Watermelon extract 104 contains citrulline, which increases the level of arginine, an amino acid that improves blood circulation in the scalp, thus making the hair grow faster and stronger.

Pineapple extract 105 obtained from fruit pulp containing vitamin C, when applied, makes hair softer, shinier, and thicker, and also inhibits hair loss. Pineapple extract 105 also inhibits inflammation on the scalp which reduces the occurrences of dermatitis, psoriasis and eczema. Lime oil 106 acts as an essential oil and reduces dandruff, promotes hair growth, and removes excessive oil from hair, thereby enhancing natural hair shine. The lime oil 106, used in the present invention, can be cold-pressed from the peel of fresh limes.

For providing additional antioxidants, natural sources of Vitamins A, C, E 107 are used to help in maintaining a healthy scalp and hair growth. Vitamins 107 further maintain the pH of the scalp by locking in the natural moisture of the hair, and by increasing capillary circulation in the scalp. Flavonoids 108 derived from natural plants, such as chrysanthemum or wormwood plants, act as an active ingredient of the spray 100. Flavonoids 108 also include antimicrobial, anticancer, antiviral, antiallergic and anti-inflammatory properties.

Phenolic compounds 109 can be provided from natural sources such as dehydrated green tea, corn silk or Annurca apple and are added into the spray composition 100. Phenolic compounds 109 act as antioxidants to promote hair growth and follicle growth. Ferulic acid 110 can be added to the composition 100 to provide antioxidant and anti-inflammatory powers to further aid healthy hair growth. Ferulic acid 110 can be derived from plants such as rice and oats, and acts to boost and stabilize both vitamin C and vitamin E in fighting free radical damage and collagen production. Allantoin 111, and derivatives thereof, reactivates trichogen cells and helps in achieving long, thick hairs. Orange extract 112 is a great source of antioxidant, infuses hair with vitamins, soothes irritated scalps and treats hair loss. Orange extract 112 acts as both an antiseptic and an anti-inflammatory, thereby rendering it effective for treating dandruff and other scalp issues.

The aforementioned ingredients, extracted from plants and seeds of fruits, have beneficial effects on hair growth. The oils extracted are biologically able to promote hair growth, and are excellent for cleansing, nourishing, and strengthening the hair follicle and shaft. The oils extracted are preferably organic and can be refined or unrefined depending on the amount and type of impurities present. It should be appreciated that other oils having similar physical and nutritive properties may also be used.

The hair growth composition of the present invention is safe for humans and has been demonstrated to not cause irritation to the scalp even when used for a long period of time. The composition of the spray 100 can be used effectively on both damp and dry hair, thereby eliminating the need for multiple products. It can be used as both a pre-shampoo and a post-shampoo treatment. The composition of the present invention can be used on all hair types, textures, quantities and lengths according to a user's needs and lifestyle.

In one potential embodiment, the distilled water or carrier base fluid can act as a solvent, carrier, diluent and/or dispersant for the constituents of the composition, and may allow for the uniform application of the constituents to the surface of the skin at an appropriate dilution, e.g., topical application. For example, carrier base fluids can be emulsions, lotions, creams, tonics, sprays, aerosols and the like. The carrier base fluid may also facilitate penetration of the composition into the skin.

Referring to Table 1, there is disclosed a hair loss treatment composition spray 100 composition with a number of different components in varying quantities in order to achieve a desired result. The hair loss treatment composition spray 100, in one potential embodiment, is provided as a spray solution. One such exemplary embodiment of the hair loss treatment composition spray 100 is provided as shown in Table 1.

| | Content in percentage |
|---|---|
| Component | by weight (% w/w) |
| Glycerin | 3-6 |
| Grapeseed oil | 0.3-10 |
| Watermelon extract | 3-5 |
| Pineapple extract | 2-24 |
| Lime oil | 3-15 |
| Flavonoids | 0.1-2 |
| Phenolic compounds | 0.5-1.8 |
| Ferulic acid | 0.3-2.2 |
| Allantoin | 0.5-0.8 |
| Vitamin A, C, E | 0.2-3 |
| Orange Extract | 2-14 |
| Distilled water or | An amount to make |
| carrier base fluid | the total 100 |

Table 2, there is disclosed alternative hair loss treatment composition spray 100 with a number of different components in varying quantities in order to achieve a desired result.

| | Percentage by |
|---|---|
| Component | weight (% w/w) |
| Glycerin | 4-5 |
| Grapeseed oil | 1-5 |
| Watermelon extract | 4-8 |
| Pineapple extract | 3-20 |
| Lime oil | 4-8 |
| Flavonoids | 1-2 |
| Phenolic compounds | 0.8-1.5 |
| Ferulic acid | 0.4-2.1 |
| Allantoin | 0.5-0.8 |
| Vitamin A, C, E | 1-2.5 |
| Orange Extract | 3-18 |
| Distilled water or | An amount to make |
| carrier base fluid | the total 100 |

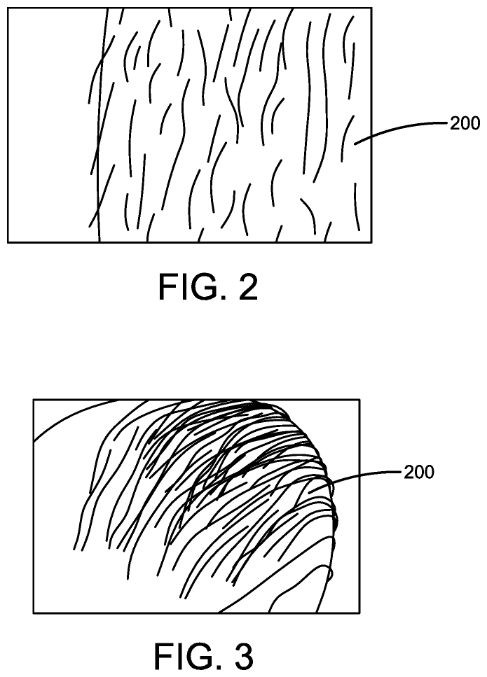

FIG. 2 illustrates an image of an individual prior to using the hair loss treatment composition spray of the present invention. The density and length of the hair of the user 200 are less than desirable, and are in need for treatment with the hair care composition 100.

FIG. 3 illustrates an image of the same individual after using the hair loss treatment composition spray of the present invention for a month. As shown, density, length and strength of the hair of the user 200 has increased by application of the hair loss treatment composition spray. The spray can be in the form of an aqueous solution, and can be used as an additional step when washing and conditioning hair.

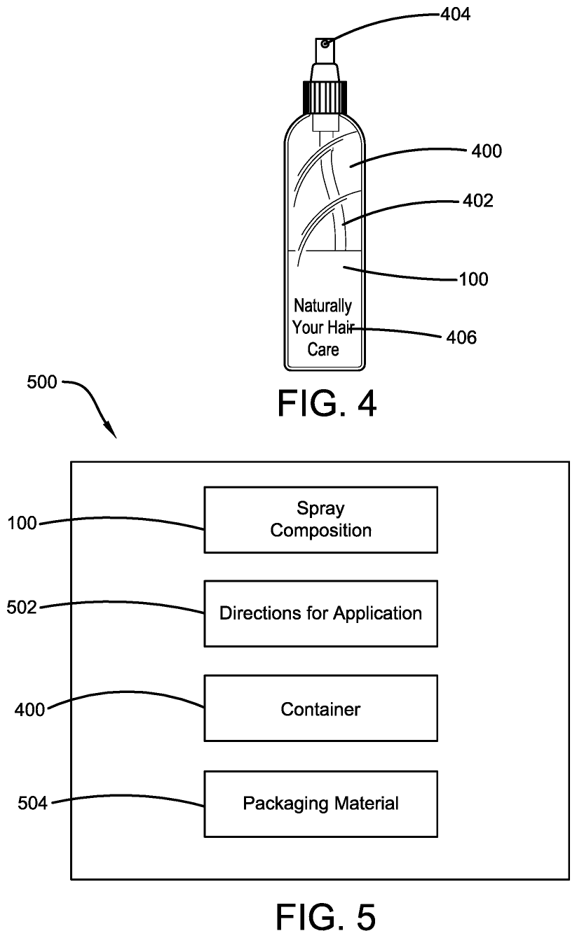

FIG. 4 illustrates an exemplary container, e.g. pump sprayer, in which the hair loss treatment composition spray can be dispensed, marketed, and sold. The container 400 contains the spray composition 100 in a liquid form which is dispensed through a nozzle spray port 404. The spray port 404 is connected to a conduit 402 through which the spray composition 100 is moved upwards for expulsion through the nozzle spray port 404.

The spray composition 100 can be packaged in any size container, for example, 4, 6, 8 and 10 ounce containers. The container 400 can also include a logo, indicia or trademark 406 along with customizable colors and fonts, embroidery and prints and/or other images on the surface.

The container 400 may be of any suitable shape and size, including but not limited to, rectangular, cubic, pyramidal, cylindrical, spherical or any other shape. The containers shown in the figures, and described in the embodiments, are not limited to any particular size or shape and can include aerosol cans from which the spray 100 emerges as an aerosol mist.

FIG. 5 illustrates components of a kit in which the hair loss treatment composition spray can be marketed and commercially available. As shown, the hair loss treatment composition kit 500 includes a packaging material 504 in the form of a box in which the container 400 is secured. The packaging material 504 can have a logo, trademark and ingredients on the surface. A direction for application document 502 can also be included in the kit 500 to help a user in applying the spray composition 100. The container 400 contains the spray composition 100 in a sealed manner.

The methods of preparing the spray composition 100 utilizes two containers, namely a first container having a mixture of the oils, glycerin, and fruit extracts, and a second container having a mixture of flavonoids, phenolic compounds, ferulic acid and allantoin. The ingredients of the first container and the ingredients of the second container, respectively, can be held as a first concentrate and a second concentrate. The contents of both containers can be mixed together along with the distilled water (i.e. carrier base fluid) and the natural vitamins A, C, E to form the spray composition.

The composition of the present invention is useful as a pharmaceutical or an ayurvedic product and functions as a hair care product for people of all ages, and for any hair color and type. The all-natural hair spray is designed to promote hair strength, durability, growth and offers a treatment to improve overall hair health.

The embodied compositions, when applied to an individual's hair, is effective in maintaining nourishment of the hair both internally and externally. The embodied compositions help fill in the nutritional gaps that may be caused by the illness or malnourishment of the scalp and hair.

The preferred embodiments of the present invention provide a hair care composition that is applied as a spray to the hair and the scalp. The spray 100 provides nourishment to the hair follicle, thereby promoting hair health. More particularly, the spray 100 reverses hair loss and increases the rate of hair growth with regular use. The containers to store the spray treatment 100 may be made available in different sizes and colors to accommodate user needs and preferences. Exact size, measurement, construction and design specifications can vary upon manufacturing or material used.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “hair loss treatment composition spray”, “spray composition”, “spray”, and “spray treatment” are interchangeable and refer to the hair loss treatment composition spray 100 of the present invention.

Notwithstanding the forgoing, the hair loss treatment composition spray 100 of the present invention can contain any additional ingredient known in the art without affecting the overall concept of the invention, provided that it accomplishes the above-stated objectives. One of ordinary skill in the art will appreciate that the ingredients and composition of the hair loss treatment composition spray 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for treating and managing hair loss, comprising the steps of:
administering to a subject experiencing hair loss a therapeutically effective amount of a composition, the composition comprising:
a glycerin, wherein the glycerin comprises from about 3% to about 6% by weight of the composition;
a grapeseed oil, wherein the grapeseed oil comprises from about 0.3% to about 10% by weight of the composition;
a fruit extract, wherein the fruit extract comprises from about 2% to about 24% by weight of the composition;
a lime oil, wherein the lime oil comprises from about 3% to about 15% by weight of the composition;
a natural vitamin, wherein the natural vitamin comprises from about 0.2% to about 3% by weight of the composition;
a flavonoid, wherein the flavonoid comprises from about 0.1% to about 2% by weight of the composition;
a phenolic compound, wherein the phenolic compound comprises from about 0.5% to about 1.8% by weight of the composition;
a ferulic acid, wherein the ferulic acid comprises from about 0.3% to about 2.2% by weight of the composition; and
an allantoin, wherein the allantoin comprises from about 0.5% to about 0.8% by weight of the composition; and
rinsing the composition from the subject.

2. The method of claim 1, wherein the glycerin comprises from about 4% to about 5% by weight of the composition.

3. The method of claim 1, wherein the grapeseed oil comprises from about 1% to about 5% by weight of the composition.

4. The method of claim 1, wherein the fruit extract comprises from about 3% to about 20% by weight of the composition.

5. The method of claim 1, wherein the lime oil comprises from about 4% to about 8% by weight of the composition.

6. The method of claim 1, wherein the natural vitamin comprises from about 1% to about 2.5% by weight of the composition.

7. The method of claim 1, wherein the flavonoids comprise from about 1% to about 2% by weight of the composition.

8. The method of claim 1, wherein the phenolic compounds comprise from about 0.8% to about 1.5% by weight of the composition.

9. The method of claim 1, wherein the ferulic acid comprises from about 0.4% to about 2.1% by weight of the composition.

10. The method of claim 1, wherein the fruit extract comprises at least one fruit selected from a group consisting of: a watermelon, an orange and a pineapple.

11. The method of claim 1, wherein the natural vitamins comprise at least one vitamin selected from a group consisting of a Vitamin A, a Vitamin C and a Vitamin E.

12. The method of claim 1, wherein the composition further comprises a carrier fluid, and further wherein the carrier fluid is a distilled water.

13. The method of claim 1, wherein the composition is in the form of a liquid for dispensing from a pump sprayer.

14. The method of claim 1, wherein the composition further comprises a carrier fluid, wherein the carrier fluid is selected from a group consisting of an emulsion, a lotion, a cream, a tonic, a spray and an aerosol.

15. A composition for treating and managing hair loss in an individual, the composition comprising:
a glycerin, wherein the glycerin comprises from about 4% to about 5% by weight of the composition;
a grapeseed oil, wherein the grapeseed oil comprises from about 1% to about 5% by weight of the composition;
a fruit extract, wherein the fruit extract comprises from about 3% to about 20% by weight of the composition;
a lime oil, wherein the lime oil comprises from about 4% to about 8% by weight of the composition;
a natural vitamin, wherein the natural vitamin comprises from about 1% to about 2.5% by weight of the composition;
a flavonoid, wherein the flavonoid comprises from about 1% to about 2% by weight of the composition;
a phenolic compound, wherein the phenolic compound comprises from about 0.8% to about 1.5% by weight of the composition;
a ferulic acid, wherein the ferulic acid comprises from about 0.4% to about 2.1% by weight of the composition; and
an allantoin, wherein the allantoin comprises from about 0.5% to about 0.8% by weight of the composition.

16. The composition of claim 15, wherein the fruit extract comprises at least one fruit selected from a group consisting of: a watermelon, an orange and a pineapple.

17. The composition of claim 15, wherein the natural vitamin comprises at least one vitamin selected from a group consisting of: a Vitamin A, a Vitamin C and a Vitamin E.

18. The composition of claim 15 further comprising a distilled water.

19. The composition of claim 15, wherein the composition is in a form selected from a group consisting of an emulsion, a lotion, a cream, a tonic, a spray and an aerosol.

20. A composition for treating and managing hair loss in an individual, the composition comprising:
a glycerin;
a grapeseed oil;
a fruit extract;
a lime oil;
a natural vitamin;
a flavonoid;
a phenolic compound;
a ferulic acid;
a distilled water; and
an allantoin, wherein the composition is in a select one of the following forms: an emulsion, a lotion, a cream, a tonic, a spray and an aerosol.
